(19) United States
(12) Patent Application Publication
(10) Pub. No.: US 2024/0009303 A1
(43) Pub. Date: Jan. 11, 2024

(54) COMBINATION VACCINE FOR PROTECTING SWINE AGAINST VARIOUS DISORDERS

(71) Applicant: Intervet Inc., Madison, NJ (US)

(72) Inventors: Sietske Kooijman, BOXMEER (NL); Ruud Philip Antoon Maria Segers, Boxmeer (NL); Maarten Hendrik Witvliet, Boxmeer (NL)

(73) Assignee: Intervet Inc., Madison, NJ (US)

(21) Appl. No.: 18/250,106

(22) PCT Filed: Oct. 28, 2021
(86) PCT No.: PCT/EP2021/079912

§ 371 Date: Apr. 21, 2023

(30) Foreign Application Priority Data
Oct. 29, 2020 (EP) 20204555.5

(51) Int. Cl. A61K 39/295; A61P 31/12; A61K 39/02; A61K 39/12; A61P 31/04; A61K 39/385

(52) U.S. Cl. CPC A61K 39/295; A61P 31/12; A61K 39/0241; A61K 39/12; A61P 31/04; A61K 39/385; A61K 2039/552

(57) ABSTRACT

The present invention pertains to a vaccine comprising in combination non-replicating immunogen of porcine circo virus type 2 (PCV2), non-replicating immunogen of Mycoplasma hyopneumoniae and conjugated deoxynivalenol (DON) for protecting swine against an infection with porcine circo virus type 2, an infection with Mycoplasma hyopneumoniae and DON induced mycotoxicosis.

### GENERAL FIELD OF THE INVENTION

The invention in general pertains to the field of swine health. Swine are prone to many pathogenic micro-organisms and adverse events such as intoxication by mycotoxins present in animal feed. Control in these respects is commonly done by farm and feed management, treatment with pharmaceuticals such as anti-viral drugs and antibiotics, prophylactic treatment using vaccines, treating the feed with toxin binders etc. For example, almost all swine are prone to infection with porcine circo virus type 2 (PCV2 or PCV-2) and Mycoplasma hyopneumoniae (Mhyo), as well as mycotoxicosis induced by the mycotoxin deoxynivalenol (DON) present in animal feed.

### BACKGROUND OF THE INVENTION

PCV-2 is linked to the post-weaning multisystemic wasting syndrome (PMWS) observed in young pigs. This disease was encountered for the first time in Canada in 1991. The clinical signs and pathology were first published in 1996, and include progressive wasting, dyspnea, tachypnea, and occasionally icterus and jaundice. Nayar et al., Can. Vet. J. Volume 38, June 1997 detected porcine circo virus in pigs with clinical symptoms of PMWS and concluded that a PCV, other than the known PCV recognized as a natural inhabitant of PK-15 cells, could be linked to PMWS. Later publications (Harnel et al., J. Virol., 72(6), 5262-5267, 1998; Meehan et al., J. Gen. Virol., 79, 2171-2179, 1998) confirmed these findings, and it was proposed (Meehan et al., supra) to refer to the new pathogenic PCV as PCV-2, whereas the original PK-15 cell culture isolate (Tischer et al., Nature 295, 64-66, 1982), should be referred to as PCV-1. PCV-2 is a small (17-22 nm) icosahedral non-enveloped virus containing a circular single stranded DNA genome. The length of the PCV-2 genome is about 1768 bp. PCV-2 isolates originating from different regions in the world seem to be closely related to each other and display about 95 to 99% nucleotide sequence identities (Fenaux et al., J. Clin. Micorbiol., 38(7), 2494-2503, 2000). ORF2 of PCV encodes the capsid protein of the virus. The ORF2 gene of PCV 2 encodes a protein of about 233 amino acids. The ORF 2 gene of all PCV-2 isolates share 91-100% nucleotide sequence identity and 90-100% deduced amino acid sequence identity.

Mycoplasma hyopneumoniae is a species of bacteria known to cause the disease Porcine Enzootic Pneumonia, a highly contagious and chronic disease affecting pigs. Mhyo is small in size (400-1200 nm), has a small genome (893-920 kilo-base pairs (kb)) and lacks a cell wall. Mhyo attaches to the cilia of epithelial cells in the lungs of swine. They cause cilia to stop beating, clumping and loss of cilia, eventually leading to epithelial cell death. This is the source of the lesions found in the lungs of pigs with porcine enzootic pneumonia. This damage impedes normal ciliary clearance and often secondary infections develop. This causes a significant reduction in the growing weight of the animals. Losses in the U.S.A. have been previously estimated to be up to 1 billion dollars per annum. Porcine enzootic pneumonia is endemic worldwide and Mhyo is present in almost every pig herd. The immune response induced by the presence of Mhyo in pigs is slow and ineffective. Treatment of this disease is therefore of the utmost importance but is limited to antibiotics, which are currently only partly effective as they do not completely remove the infection. Vaccines have been found to reduce the severity of the disease but do not completely prevent the disease from occurring in infected pigs.

Another pathogen that is widespread under swine world-wide is Lawsonia intracellularis. This bacterium may cause proliferative enteropathy, also known as ileitis, which is a common enteric disease of post-weaned pigs worldwide. The characteristic lesion is a proliferation of immature enterocytes in the ileal intestinal crypts; these cells usually contain the causative bacteria within their apical cytoplasm. At autopsy, histologic lesions can be confirmed as Lawsonia-positive by visualization of 1.5-2.5 μm long, vibrioid shaped bacteria especially in enterocytes, but also often within macrophages located in the lamina propria between crypts, and in mesenteric lymph nodes. Clearance of the bacteria from the enterocytes leads to resolution of the associated proliferative lesions, indicating a direct local effect of the bacteria on the crypts. The presence of Lawsonia intracellularis in these lesions has been demonstrated using PCR, both in animals manifesting disease as in animals manifesting only subclinical infection. Clinical cases are usually present in the grower-finisher period; in some older finisher pigs an acute hemorrhagic form has been recorded.

Fungi in general cause a broad range of diseases in swine, involving parasitism of organs and tissues as well as allergenic manifestations. However, other than poisoning through ingestion of non-edible mushrooms, fungi can produce mycotoxins and organic chemicals that are responsible for various toxic effects referred to as mycotoxicosis. This disease is caused by exposure to mycotoxins, pharmacologically active compounds produced by filamentous fungi contaminating foodstuffs or animal feeds. Mycotoxins are secondary metabolites not critical to fungal physiology, that are extremely toxic in minimum concentrations to vertebrates upon ingestion, inhalation or skin contact. About 400 mycotoxins are currently recognized, subdivided in families of chemically related molecules with similar biological and structural properties. Of these, approximately a dozen groups regularly receive attention as threats to animal health. Examples of mycotoxins of greatest public interest and agroeconomic significance include aflatoxins (AF), ochratoxins (OT), trichothecenes (T; including DON), zearalenone (ZEN), fumonisins (F), tremorgenic toxins, and ergot alkaloids. Mycotoxins have been related to acute and chronic diseases, with biological effects that vary mainly according to the diversity in their chemical structure, but also with regard to biological, nutritional and environmental factors. The pathophysiology of mycotoxicoses is the consequence of interactions of mycotoxins with functional molecules and organelles in the animal cell, which may result in carcinogenicity, genotoxicity, inhibition of protein synthesis, immunosuppression, dermal irritation, and other metabolic perturbations. In sensitive animal species, mycotoxins may elicit complicated and overlapping toxic effects. Mycotoxicoses are not contagious, nor is there significant stimulation of the immune system.

Deoxynivalenol, also known as vomitoxin. is a type B trichothecene which is present predominantly in grains such as wheat, barley, oats, rye, and corn, but also in rice, sorghum, and triticale. The occurrence of deoxynivalenol is associated primarily with Fusarium graminearum (Gibberella zeae) and Fusarium culmorum, both of which are important plant pathogens which cause fusarium head blight in wheat and gibberella or fusarium ear blight in corn. A direct relationship between the incidence of fusarium head blight and contamination of wheat with deoxynivalenol has been established. The incidence of fusarium head blight is strongly associated with moisture at the time of flowering, and the timing of rainfall, rather than the amount, is the most critical factor. Furthermore, DON contents are significantly affected by the susceptibility of cultivars towards Fusarium species, previous crop, tillage practices, and fungicide use. Fusarium graminearum grows optimally at a temperature of 25° C., whereas Fusarium culmorum grows optimally at 21° C. Fusarium graminearum therefore being the more common species occurring in warmer climates.

DON has been implicated in incidents of mycotoxicoses in both humans and farm animals. The toxin belongs to the class of trichothecenes which are strong inhibitors of protein synthesis. Exposure to DON causes the brain to decrease its uptake of the amino acid tryptophan and, in turn, its synthesis of serotonin. Reduced levels of serotonin are believed to be responsible for the anorexic effects of DON. Irritation of the gastrointestinal tract may also play a role in reducing feed intake, and may also partially explain the high incidence of paraesophageal stomach ulcers observed in sows during feed refusal.

Prophylactic treatment of DON induced mycotoxicosis is currently restricted to good agricultural practice to reduce mycotoxins production on crop and control programs of food and feed commodities to ensure that mycotoxin levels remain below certain limits. Treatment with drugs or antibiotics has little or no effect on the course of the disease. To date no human or animal vaccine is available for combating mycotoxicoses.

This is different for the various above identified pathogens, vaccines against these pathogens are commonly known. A conventional vaccine to prophylactically treat animals, in particular pigs, against an infection with PCV 2, may be based on whole inactivated PCV-2 virus as (non-replicating) immunogen. Also, in the art it has been shown that the ORF2 encoded capsid protein (e.g. when recombinantly expressed) is suitable as a subunit immunogen of porcine circo virus type 2 for use in an adequate vaccine. This can be understood since this subunit in a circulatory system, shows up the same way as the virus itself (it forms virus-like particles), essentially differing only in the fact that the DNA and non-structural proteins are not present inside the capsid. In the art several vaccines against PCV2 are commercially available. Porcilis® PCV (available from MSD Animal Health, Boxmeer, The Netherlands) is a vaccine for protection of pigs against porcine circo virus type 2, for use in pigs from three weeks and older. When given as a two-shot (two dose) vaccine, the duration of immunity (DOI) is 22 weeks, almost completely covering the fattening period of pigs. Ingelvac CircoFlex® (available from Boehringer Ingelheim, Ingelheim) is a vaccine for protection of pigs against porcine circo virus type 2, for use in pigs from two weeks and older. It is registered as a one-shot (one dose) vaccine only. Circovac® (available from Ceva Sante Animale, Libourne, France) is a vaccine for protection of pigs against porcine circo virus type 2, for use in pigs three weeks and older. Suvaxyn® PCV (available from Zoetis, Capelle a/d IJssel, The Netherlands) is a vaccine for protection of pigs against porcine circo virus type 2, for use in pigs from three weeks and older. Other PCV2 vaccines are described for example in WO2007/028823, WO 2007/094893 and WO2008/076915. These vaccines all have in common that they comprise the ORF2 capsid protein of PCV2.

Regarding Mycoplasma hyopneumoniae many commercial vaccines exist, and these are routinely used in the majority of commercial swine farming operations. Generally, these vaccines comprise non-replicating immunogens of Mhyo such as subunit proteins and/or bacterins which are typically administered by parenteral injection. Some examples are: RespiSure® (Zoetis), Ingelvac® M. hyo, and MycoFLEX® (Boehringer Ingelheim), Stellamune® Mycoplasma (Elanco Animal Health), Fostera® PCV MH (Zoetis) and M+Pac® and Porcilis® M Hyo (both available from MSD Animal Health).

Vaccines to combat Lawsonia intracellularis by inducing active protection are commercially available and described in the art. These vaccines are available under the tradenames Enterisol® Ileitis (Boehringer Ingelheim Vetmedica, USA) which is a live attenuated vaccine, and Porcilis® Ileitis (Merck Animal Health, USA), or Porcilis® Lawsonia (MSD Animal Health, The Netherlands) which are both vaccines comprising non-replicating immunogen of Lawsonia intracellularis in the form of a bacterin.

### Object of the Invention

It is an object of the invention to provide a composition that is able to protect a swine against an infection with porcine circo virus type 2 (PCV2 or PCV-2) and Mycoplasma hyopneumoniae (Mhyo), as well as mycotoxicosis induced by the mycotoxin deoxynivalenol (DON) in one go.

### SUMMARY OF THE INVENTION

In order to meet the object of the invention a vaccine has been devised that comprises in combination a non-replicating immunogen of porcine circo virus type 2 (PCV2), a non-replicating immunogen of Mycoplasma hyopneumoniae and a conjugated deoxynivalenol (DON). This vaccine appears to be suitable for protecting swine against an infection with porcine circo virus type 2, an infection with Mycoplasma hyopneumoniae and DON induced mycotoxicosis in one go, all very widespread disorders among swine. Although a combination vaccine against PCV2 and Mhyo has been known in the art, it was surprising to find that conjugated deoxynivalenol is suitable for use as a vaccine to protect an animal against DON induced mycotoxicosis. It was found that there was no particular need (although not excluded by the claims) to convert the DON into a toxoid, the conjugated toxin appeared to be safe for the treated host animal. Also, it was surprising to see that the immune response induced was strong enough to actually protect the vaccinated animal against mycotoxicosis after oral ingestion of DON post treatment. Active protection by inducing an immune response against the mycotoxin has not been shown in the art for any mycotoxin, let alone for the highly abundant and extremely toxic compound deoxynivalenol. Another surprise was to see that the three components of the vaccine do not negatively interfere. With the combination vaccine, an adequate immune response can be raised against each of the antigens. Such a response is protective against the pathogens PCV2 and Mhyo as known in the art, and now proven for the first time to be protective against DON intake.

The invention also pertains to a vaccine comprising in combination a non-replicating immunogen of porcine circo virus type 2 (PCV2), a non-replicating immunogen of Mycoplasma hyopneumoniae and a conjugated deoxynivalenol (DON) for use in a method of protecting a swine against an infection with porcine circo virus type 2, an infection with Mycoplasma hyopneumoniae and DON induced mycotoxicosis.

The invention further pertains to a kit-of-parts comprising in combination a first composition comprising in combination a non-replicating immunogen of porcine circo virus type 2, a non-replicating immunogen of Mycoplasma hyopneumoniae (the term “composition” not excluding that it pertains to two separate containers of which the contents are to be mixed before administration) and a second composition comprising a conjugated deoxynivalenol (DON). The kit-of-parts may contain an instruction how to use the two compositions for administration to a swine in one go, for example by mixing them preceding the actual administration, or by associated non-mixed use using an administration device with two separate administration nozzles/barrels such as the IDAL® 3G TWIN (MSD Animal Health).

### Definitions

A vaccine is a pharmaceutical composition that is safe to administer to a subject animal, and is able to induce protective immunity in that animal against a pathogenic micro-organism or compound, i.e. to induce a successful prophylactic treatment as defined here below.

Non-replicating immunogen of a pathogen is any substance or compound corresponding to the pathogen, other than the live replicating pathogen as a whole (either in wild type of attenuated form), against which pathogen an immunological response is to be elicited, such that the corresponding virulent pathogen or one or more of its virulence factors will be recognized by the host's immune system as a result of this immune response and are ultimately at least partly neutralized. Typical examples of non-replicating immunogens are killed whole pathogens (which term includes these pathogens in lysed form) and subunits of these pathogens such as capsid proteins, surface expressed molecules (for example recombinantly expressed proteins or lipopolysaccharides) and excreted molecules such as toxins. This group of immunogens has in common that they typically elicit a humoral immune response.

A bacterin is a suspension of killed bacteria, e.g. obtained by concentration of a bacterial culture that is subsequently inactivated with a chemical agent such as binary ethylenimine (BEI), chlorocresol, formalin, or for example by UV light or other types of inactivation

Prophylactic treatment, for example against an infection with a pathogen or against another adverse event, is aiding in preventing, ameliorating or curing the infection with that pathogen (or a disorder arising from that infection) or aiding in preventing, ameliorating or curing the said event, wherein the treatment takes place before challenge with the pathogenic pathogen or the ocurrence of the event respectively.

Mycotoxicosis is the disease resulting from exposure to a mycotoxin. The clinical signs, target organs, and outcome depend on the intrinsic toxic features of the mycotoxin and the quantity and length of exposure, as well as the health status of the exposed animal.

To protect against mycotoxicosis means to prevent or decrease one or more of the negative physiological effects of the mycotoxin in the animal, such as a decrease in average daily weight gain.

Deoxynivalenol (abbreviated DON, also known as vomitoxin or VOM) is a mycotoxin produced by the fungus Fusarium graminearum, which causes Fusarium head blight (FHB), or scab, of small grains. DON can cause feed refusal and vomiting. The molecular formula of the basic compound is C₁₅H₂₀O₆.

A conjugated molecule is a molecule to which an immunogenic compound is coupled through a covalent bond. Typically, the immunogenic compound is a (large) protein such as KLH, BSA or OVA.

An adjuvant is non-specific immunostimulating agent. In principal, each substance that is able to favor or amplify a particular process in the cascade of immunological events, ultimately leading to a better immunological response (i.e. the integrated bodily response to an antigen, in particular one mediated by lymphocytes and typically involving recognition of antigens by specific antibodies or previously sensitized lymphocytes), can be defined as an adjuvant. An adjuvant is in general not required for the said particular process to occur, but merely favors or amplifies the said process.

### Further Embodiments of the Invention

In a further embodiment of the vaccine according to the invention the non-replicating immunogen of PCV2 is the ORF2 protein of PCV2. This immunogen is proven to elicit an adequate protective immune response against the PCV2 virus and appears to be suitable for use in the present combination vaccine, i.e. to allow a concurrent stimulation of the immune system will still being safe for the subject swine. The immunogen may be recombinantly expressed ORF2 protein of PCV2, such as for example baculovirus expressed ORF2 protein.

In yet another embodiment the non-replicating immunogen of Mycoplasma hyopneumoniae is a Mycoplasma hyopneumoniae bacterin, which may comprise killed whole Mycoplasma hyopneumoniae. A bacterin of Mhyo, in particular when still containing whole cells, has shown to be adequate for use in the present combination vaccine.

In again another embodiment the conjugated DON comprises DON conjugated to a protein having a molecular mass above 10.000 Da. Such proteins, in particular keyhole limpet hemocyanin (KLH) and ovalbumin (OVA), have been found to be able and induce an adequate immune response in swine and other animals. A practical upper limit for the protein might be 100 MDa. Above this limit physical disadvantages may appear.

In yet again another embodiment the vaccine comprises non-replicating immunogen of Lawsonia intracellularis, in particular killed whole cells of Lawsonia intracellularis, such as known for example form the commercial vaccine Porcilis Ileitis (available through Merck Animal Health) and Porcilis Lawsonia (available through MSD Animal Health).

In a further embodiment of the combination vaccine for use in a method of protecting a swine against an infection with porcine circo virus type 2, an infection with Mycoplasma hyopneumoniae and DON induced mycotoxicosis, the vaccine is systemically administered to the swine. Although local administration, for example via mucosal tissue in the gastro-intestinal tract (oral or anal cavity) or in the eyes (for example when immunising chickens) is known to be an effective route to induce an immune response in various animals, it was found that systemic administration leads to an adequate immune response for protecting animals against all three disorders. It was found in particular that effective immunisation can be obtained upon intramuscular and/or intradermal administration.

The age of administration is not believed to be critical, although it is preferred that the administration takes place before the swine loses its maternal immunity and is able to ingest feed contaminated with substantial amounts of DON. Hence a preferred age at the time of administration of 6 weeks or younger. Further preferred is an age of 4 weeks or younger, such as for example an age of 1-3 weeks.

The vaccine according to the invention may be administered to the animal at least once or twice. Although many animals (in particular swine chickens, ruminants) in general are susceptible for immunisation by only one shot of an immunogenic composition, it is believed that for economic viable protection against DON two shots are preferred. The first or second shot may be a monovalent vaccine containing only conjugated DON while the other shot is done with the combination vaccine. This is based on the fact that for protection against PCV2 and Mhyo, one shot has proven to provide effective protection throughout the life span of a typical swine. However, it is also foreseen that an animal receives a prime and a boost vaccination with the novel combination vaccine. The time period between the two shots of the vaccines can be anything between 1 week and 1-2 years. For young animals it is believed that a regime of a prime immunisation, for example at 1-3 weeks of age, followed by a booster administration 1-4 weeks later, typically 1-3 weeks later, such as 2 weeks later, will suffice. Older animals may need a booster administration every few months (such as 4, 5, 6 months after the last administration), or on a yearly or biannual basis as is known form other commercially applied immunisation regimes for animals.

The combination vaccine may comprise an adjuvant in addition to the three antigens. An adjuvant may be used if the antigens on themselves are not able to induce an immune response to obtain a predetermined level of protection. Although conjugate molecules with carrier molecules such as KLH or BSA are known to be able to sufficiently stimulate the immune system without an additional adjuvant, it may be advantageous to use an additional adjuvant. This could take away the need for a booster administration or prolong the interval for the administration thereof. This all depends on the level of protection needed in a specific situation. Type of adjuvants believed to be particularly suitable for use with the current vaccine are oil-in-water emulsions (such as for example based on mineral oil, shark liver oil, vitamin E acetate etc), solutions of Carbopol®, and alhydrogel and other aluminium containing adjuvant systems.

The invention will now be further explained using the following examples.

### EXAMPLES

In a first series of experiments (described in Examples 1 through 3 below), the efficacy of a monovalent DON vaccine was tested for safety and efficacy. Thereafter, the efficacy of the trivalent vaccine according to the invention was tested accordingly (Example 4).

### Example 1

### Immunisation Challenge Experiment Using Conjugated DON

### Objective

The objective of this study was to evaluate the safety and efficacy of conjugated deoxynivalenol to protect swine against mycotoxicosis due to DON ingestion. To examine this, pigs were immunised twice with DON-KLH before being challenged with toxic DON. Different routes of immunisation were used to study the influence of the route of administration.

### Study Design

Forty 1 week old pigs derived from 8 sows were used in the study, divided over 5 groups. Twenty-four piglets of group 1-3 were immunised twice at 1 and 3 weeks of age. Group 1 was immunised intramuscularly (IM) at both ages. Group 2 received an IM injection at one week of age and an oral boost at three weeks of age. Group 3 was immunised intradermally (ID) two times. From 5½ weeks of age groups 1-3 were challenged during 4 weeks with DON administered orally in a liquid. Group 4 was not immunised but was only challenged with DON as described for groups 1-3. Group 5 served as a control and only received a control fluid, from the age of 5.5 weeks for 4 weeks.

The DON concentration in the liquid formulation corresponded to an amount of 5.4 mg/kg feed. This corresponds to an average amount of 2.5 mg DON per day. After four weeks of challenge all animals were post-mortem investigated, with special attentions for the liver, kidneys and the stomach. In addition, blood sampling was done at day 0, 34, 41, 49, 55, 64 (after euthanasia) of the study, except for group 5 of which blood samples were taken only at day 0, 34, 49, and directly after euthanasia.

### Test Articles

Three different immunogenic compositions were formulated, namely Test Article 1 comprising DON-KLH at 50 μg/ml in an oil-in-water emulsion for injection (X-solve 50, MSD AH, Boxmeer) which was used for IM immunization; Test Article 2 comprising DON-KLH at 50 μg/ml in a water-in-oil emulsion (GNE, MSD AH, Boxmeer) which was used for oral immunization and Test Article 3 comprising DON-KLH at 500 μg/ml in an oil-in-water emulsion for injection (X-solve 50) for ID immunisation.

The challenge deoxynivalenol (obtained from Fermentek, Israel) was diluted in 100% methanol at a final concentration of 100 mg/ml and stored at <−15° C. Prior to usage, DON was further diluted and supplied in a treat for administration.

### Inclusion Criteria

Only healthy animals were used. In order to exclude unhealthy animals, all animals were examined before the start of the study for their general physical appearance and absence of clinical abnormalities or disease. Per group piglets from different sows were used. In everyday practice all animals will be immunised even when pre-exposed to DON via intake of DON contaminated feed. Since DON as such does not raise an immune response, it is believed that there is no principle difference between animals pre-exposed to DON and naïve with respect to DON.

### Results

None of the animals had negative effects associated with the immunisation with DON-KLH. The composition thus appeared to be safe.

All pigs were serologically negative for titres against DON at the start of the experiment, During the challenge the groups immunised intramuscular (Group 1) and intradermally (Group 3) developed antibody responses against DON as measured by ELISA with native DON-BSA as the coating antigen. Table 1 depicts the average IgG values on 4 time points during the study with their SD values. Both Intramuscular immunisation and Intradermal immunisation induced significant titres against DON.

TABLE 1
IgG titres

| | group 1 | group 2 | group 3 | group 4 | Group 5 |
|---|---|---|---|---|---|
| T = 0 | <4.3 | <4.3 | <4.3 | <4.3 | <4.3 |
| T = 35 | 11.2 | 4.86 | 9.99 | 4.3 | 4.19 |
| T = 49 | 9.56 | 4.64 | 8.81 | 4.71 | 3.97 |
| T = 64 | 8.48 | 4.3 | 7.56 | 4.3 | 3.31 |

As depicted in Table 2 all immunised animals, including the animals in Group 2 that showed no significant anti-DON IgG titre increase, showed a significant higher weight gain during the first 15 days compared to the challenge animals. With respect to the challenged animals, all animals gained more weight over the course of the study.

TABLE 2
weight analysis

| | | | | | Average additional |
|---|---|---|---|---|---|
| | | | | | weight gain |
| | | | | | compared to |
| | | | weight | weight | challenge |
| | ADG1¹ | ADG² | begin | end | animals (grams) |
| group 1 | 0.67 | 0.80 | 11.63 | 32.29 | +1060 |
| group 2 | 0.64 | 0.79 | 12.31 | 32.13 | +760 |
| group 3 | 0.58 | 0.82 | 12.88 | 32.25 | +310 |
| group 4 | 0.54 | 0.81 | 12.69 | 31.75 | 0 |
| group 5 | 0.57 | 0.80 | 11.63 | 31.08 | +390 |

¹average daily weight gain over the first 15 days of the challenge
²average daily weight gain over the last 13 days of the challenge

The condition of the small intestines (as determined by the villus/crypt ratio in the jejunum) was also monitored. In table 3 the villus/crypt ratio is depicted. As can be seen, the animals in group 3 had an average villus crypt/crypt ratio comparable to the healthy controls (group 5), while the non-immunised, challenged group (group 4) had a much lower (statistically significant) villus crypt ratio. In addition, group 1 and group 2, had a villus/crypt ratio which was significantly better (i.e. higher) compared to the non-immunised challenge control group. This indicates that the immunisation protects against the damage of the intestine, initiated by DON.

TABLE 3
villus/crypt ratio

| | group 1 | group 2 | group 3 | group 4 | group 5 |
|---|---|---|---|---|---|
| average | 1.57 | 1.41 | 1.78 | 1.09 | 1.71 |
| STD | 0.24 | 0.22 | 0.12 | 0.10 | 0.23 |

The general condition of other organs was also monitored, more specifically the liver, the kidneys and the stomach. It was observed that all three test groups (groups 1-3) were in better health than the non-immunised challenge control group (group 4). In table 4 a summary of the general health data is depicted. The degree of stomach ulcer is reported from − (no prove of ulcer formation) to ++ (multiple ulcers). The degree of stomach inflammation is reported from − (no prove of inflammation) to ++/− (initiation of stomach inflammation).

TABLE 4
General health data

| | Liver | Stomach | Stomach | |
|---|---|---|---|---|
| | colour | ulcer | inflammation | Kidneys |
| Group 1 | Normal-yellow | − | − | Pail |
| Group 2 | Normal | +/−− | − | Normal |
| Group 3 | Normal | +/− | +/−− | Normal |
| Group 4 | Pail | ++ | ++/− | Pail |
| Group 5 | Normal | + | ++/− | Normal |

### Example 2

### Effect of Immunisation on DON Levels

### Objective

The objective of this study was to evaluate the effects of immunization with a DON conjugate on the toxicokinetics of DON ingestion. To examine this, pigs were immunised twice with DON-KLH before being fed toxic DON.

### Study Design

Ten 3 week old pigs were used in the study, divided over 2 groups of 5 pigs each. The pigs in Group 1 were immunised IM twice at 3 and 6 weeks of age with DON-KLH (Test Article 1; example 1). Group 2 served as a control and only received a control fluid. At the age of 11 weeks the animals were each administered DON (Fermentek, Israel) via a bolus at a dose of 0.05 mg/kg which (based on the daily feed intake) resembled a contamination level of 1 mg/kg feed. Blood samples of the pigs were taken juts before DON administration and 0.25, 0.5, 0.75, 1, 1.5, 2, 3, 4, 6, 8, and 12 h post DON administration.

### Inclusion Criteria

Only healthy animals were used.

### Analysis of DON in Plasma

Plasma analysis of unbound DON was done using a validated LC-MS/MS method on an Acquity® UPLC system coupled to a Xevo® TQ-S MS instrument (Waters, Zellik, Belgium). The lower limit of quantification of DON in pig plasma using this method is 0.1 ng/ml.

### Toxicokinetic Analysis

Toxicokinetic modeling of the plasma concentration-time profiles of DON was done by noncompartmental analysis (Phoenix, Pharsight Corporation, USA). Following parameters were calculated: area under the curve from time zero to infinite (AUC_{0→∞}), maximal plasma concentration (Cₘₐₓ), and time at maximal plasma concentration (tₘₐₓ).

### Results

The toxicokinetic results are indicated in table 5 here beneath. As can be seen immunisation with DON-KLH decreases all toxicokinetic parameters. As it is unbound DON that is responsible for the exertion of toxic effects, it may be concluded that immunisation with DON-KLH will reduce the toxic effects caused by DON by reducing the amount of unbound DON in the blood of animals.

TABLE 5
Toxicokinetic parameters of unbound DON

| Toxicokinetic parameter | DON-KLH | Control |
|---|---|---|
| AUC_{0→∞} | 77.3 ± 23.6 | 187 ± 33 |
| Cₘₐₓ | 12.5 ± 2.7 | 30.8 ± 2.5 |
| tₘₐₓ | 1.69 ± 1.03 | 2.19 ± 1.07 |

### Example 3

### Serological Response Against Various DON Conjugates

### Objective

The objective of this study was to evaluate the efficacy of different conjugated deoxynivalenol products.

### Study Design

Eighteen 3 week old pigs were used in the study, divided over 3 groups of six pigs each. The pigs of group 1 were immunised twice intramuscularly at 3 and 5 weeks of age with DON-KLH (using Test Article 1 of Example 1). Group 2 was immunised correspondingly with DON-OVA. Group 3 served as a negative control. All animals were checked for an anti-DON IgG response at 3 weeks of age, 5 weeks of age and 8 weeks of age.

### Results

The serological results are indicated here below in the table in log2 antibody titre.

TABLE 6
anti-DON IgG response

| Test Article | 3 weeks | 5 weeks | 8 weeks |
|---|---|---|---|
| DON-KLH | 3.5 | 6.6 | 8.3 |
| DON-OVA | 3.3 | 3.9 | 11.8 |
| Control | 4.8 | 3.3 | 3.3 |

It appears that both conjugates are suitable to raise an anti-DON IgG response. Also, a response appears be induced by one shot only.

### Example 4

### Efficacy of Various Combination Vaccines

### Objective

The aim of the study was to determine whether it is possible to combine the vaccination against DON, with the vaccinations against PCV2, Mhyo and optionally Lawsonia intracellularis.

### Study Design

A herd of 64 one-week old piglets, derived from 12 sows, was divided over 8 groups of 8 piglets each. Groups 1 to 3 were used for intradermal (ID) vaccination, using the IDAL® (MSD Animal Health) device, in each case administering 0.2 ml per shot. The piglets from Group 1 received the monovalent DON-KLH vaccine as used in Example 1 (Test Article 3) as a positive control in a prime-boost scheme. Group 2 (denoted “PM”) received as a first vaccination a monovalent DON-KLH vaccine (same level of DON antigen as Group 1) in an oil-in-water emulsion (comprising squalene and vitamin E-acetate) and a second vaccination with a vaccine containing the three antigens of the invention, viz. non-replicating PCV2 immunogen (in this case baculovirus expressed ORF2 protein of PCV2 at the same level as in Porcilis® PCV ID), non-replicating Mhyo immunogen (in this case an Mhyo bacterin at the same level as in Porcilis® M Hyo ID ONCE), and the DON-KLH in the same adjuvant. Group 3 was the negative control for DON ID, receiving only Porcilis® PCV M Hyo at three weeks of age.

Groups 4 to 8 were used for intramuscular (IM) vaccination, using a standard hypodermic syringe, in each case administering 2 ml per shot. Group 4 was the positive control for the intramuscular vaccination receiving the monovalent DON-KLH vaccine (Example 1, Test Article 1) in X-solve 50 two times. Group 5 received as a first shot a monovalent DON-KLH vaccine adjuvanted with Emmunade (MSD Animal Health), and as a second shot DON-KLH mixed with Porcilis® PCV M Hyo and Porcilis® Lawsonia at three weeks in the same adjuvant (i.e. the commercial three-way Porcilis® PCV2 M Hyo Lawsonia combination vaccine, denoted “PML” in this application).The DON-KLH level was at the same level as in Test Article 1 of Example 1. Group 6 received the monovalent DON-KLH vaccine in X-solve 50 as a prime vaccination and a non-mixed associated combination vaccination with the same monovalent DON-KLH vaccine and the separate PML vaccine as a booster. Group 7 was the negative control group (PML alone). Group 8 was the negative control receiving a DON challenge.

In each of the above cases the first vaccination was administered in the right side of neck, when the piglets were one week of age and the second vaccination in the left side of neck, at three weeks of age. Challenge (Groups 2, 4, 5 and 8) took place as described here above in Example 1 using DON mixed with fluid. In the first two weeks of challenge the DON was administered in the mornings and in the evenings, and in the second two weeks of challenge the DON was administered in the morning, afternoon and evening. The dosing was such that in the first week the piglets receive 1 mg DON per day, in the second week they received 2 mg DON per day, in the third week they received 3 mg DON per day and in the fourth week they received 4 mg DON per day.

### Inclusion Criteria

Only healthy animals were used. In order to exclude unhealthy animals, they were examined before the start of the study (general physical appearance and absence of clinical abnormalities or disease).

### Results

None of the animals had negative effects associated with the various vaccinations. The compositions thus appeared to be safe.

All groups receiving a vaccine comprising conjugated DON seroconverted after vaccination (see Table 7). The ID titers were slightly lower than the IM titers. In the ID vaccinated groups the group with the combined vaccination with PM had higher titers than the group vaccinated with DON alone. This is despite the fact that the combined DON+PM group (ID) only received 30% of the DON dose. Also it was noted that the combined DON+PM (ID) group had slower decreasing titers compared to the DON alone (ID) group. Intramuscular it was observed that the results for the groups that received DON alone, combined with PML (mixed) and combined with PML (non-mixed) were very similar. This implies that for the serology against DON, neither combining with the other antigens, nor mixing appears to have a significant effect.

TABLE 7
DON serology (log2 titres)

| Group | T = 0 | T = 28 | T = 64 |
|---|---|---|---|
| 1 | <4.3 | 8.7 | 5.0 |
| 2 | <4.3 | 9.6 | 6.3 |
| 3 | <4.3 | <4.3 | <4.3 |
| 4 | <4.3 | 11.1 | 7.4 |
| 5 | 4.5 | 10.2 | 6.6 |
| 6 | <4.3 | 10.6 | 7.4 |
| 7 | <4.3 | <4.3 | <4.3 |
| 8 | 4.4 | 4.4 | <4.3 |

Protection against DON challenged was measured in the intestine by determining the villus/crypt ratio (see Table 8). The ID group (Group 2) had the highest ratio, this ratio was the same as the healthy controls in the study as described in Example 1. Both the monovalent DON IM group (Group 4), and the 4-way DON, PCV, Mhyo, Lawsonia group (Group 5) had significantly healthier intestine then the non-vaccinated challenged animals (Group 8). The values arrived at for the combination vaccine (Group 2 and 5) where even better than for the group that received the monovalent DON vaccine (Group 4) indicating that adequate protection against DON challenge was arrived at with both combination vaccines, independent of the vaccination route and the presence of additional Lawsonia antigen.

TABLE 8
villus/crypt ratio

| | Group 2 | Group 4 | Group 5 | Group 8 | Group 5, Example 1 |
|---|---|---|---|---|---|
| average | 1.74 | 1.50 | 1.65 | 1.27 | 1.71 |

Next to this, a reduction of stomach ulcers was observed in all vaccinated groups when compared to control Group 8. These data, and the data regarding the condition of the liver are depicted here below in Table 9.

TABLE 9
General health data

| | Stomach ulcers | Liver damage |
|---|---|---|
| Group 2 | +/−−−− | Mild |
| Group 4 | − | Normal to mild |
| Group 5 | − | Mild |
| Group 8 | +/− | Middle to heavy |

Thus, also at the level of stomach ulcers and liver damage it could be seen that the combination vaccine protected against DON challenge.

For showing protection against the other pathogens (PCV2, Mycoplasma hyopneumoniae and Lawsonia intracellularis) serology was measured for the IM groups at the end of the study. As is known for these existing antigens of existing commercial vaccines (Porcilis® range), a positive serology after IM vaccination indicates protection against infection with the corresponding pathogen after IM as well as ID vaccination with the same antigen. The results are indicated in Table 10. For Mhyo a negative/positive test was used. For PCV and Lawsonia an Elisa titre was measured.

TABLE 10
Serology against PCV, Mhyo and Lawsonia

| | Group 4 | Group 5 | Group 6 | Group 7 |
|---|---|---|---|---|
| PCV | <2.0 | 9.5 | 12.1 | 11.5 |
| Mhyo | negative | positive | positive | positive |
| Lawsonia | <3.9 | 5.2 | 5.9 | 5.8 |

The results show that each of the antigens elicited a positive serology, indicating that protection against the corresponding pathogens was arrived at.

### Conclusion

This study shows that DON vaccination is not impacted by simultaneous vaccination with non-replicating immunogens of PCV, Mhyo and Lawsonia, and also, that the conjugated DON antigen does not negate the protection that can be arrived using each of these three antigens.

1. A vaccine comprising in combination a non-replicating immunogen of porcine circo virus type 2 (PCV2), a non-replicating immunogen of Mycoplasma hyopneumoniae and a conjugated deoxynivalenol (DON) for protecting swine against an infection with porcine circo virus type 2, an infection with Mycoplasma hyopneumoniae and DON induced mycotoxicosis.

2. A vaccine according to claim 1, characterised in that the non-replicating immunogen of PCV2 is an ORF2 protein of PCV2.

3. A vaccine according to claim 1, characterised in that the non-replicating immunogen of PCV2 is recombinantly expressed ORF2 protein of PCV2.

4. A vaccine according to claim 1, characterised in that the non-replicating immunogen of Mycoplasma hyopneumoniae is a Mycoplasma hyopneumoniae bacterin.

5. A vaccine according to claim 1, characterised in that the non-replicating immunogen of Mycoplasma hyopneumoniae comprises killed whole Mycoplasma hyopneumoniae.

6. A vaccine according to claim 1, characterised in that the conjugated DON is DON conjugated to a protein having a molecular mass above 10.000 Da.

7. A vaccine according to claim 1, characterised in that the conjugated DON is DON conjugated to keyhole limpet hemocyanin (KLH) or ovalbumin (OVA).

8. A vaccine according to claim 1, characterised in that the vaccine comprises a non-replicating immunogen of Lawsonia intracellularis.

9. A vaccine according to claim 1, characterised in that the vaccine comprises killed whole cells of Lawsonia intracellularis.

10. A method of protecting a swine against an infection with porcine circo virus type 2, an infection with Mycoplasma hyopneumoniae and DON induced mycotoxicosis comprising administering to the swine a vaccine comprising a non-replicating immunogen of porcine circo virus type 2 (PCV2), a non-replicating immunogen of Mycoplasma hyopneumoniae and conjugated deoxynivalenol (DON).

11. The method according to claim 10, characterised in that the vaccine is systemically administered to the swine.

12. The method according to claim 10, characterised in that the vaccine is administered intramuscularly or intradermally to the swine.

13. The method according to claim 10, characterised in that the vaccine is administered to the swine at an age of 6 weeks or younger.

14. The method according to claim 10, characterised in that the vaccine is administered to the swine at an age of 4 weeks or younger, preferably at an age of 1-3 weeks.

15. A kit-of-parts comprising in combination a first composition comprising in combination a non-replicating immunogen of porcine circo virus type 2 (PCV2), a non-replicating immunogen of Mycoplasma hyopneumoniae, and a second composition comprising a conjugated deoxynivalenol (DON).
